(19) United States
(12) Patent Application Publication
(10) Pub. No.: US 2019/0142745 A1
(43) Pub. Date: May 16, 2019

(54) COMPOSITION USEFUL FOR THE PREPARATION OF A SUPPOSITORY

(71) Applicant: BIOCODEX, Gentilly (FR)

(72) Inventors: Anne BENOIST, Rungis (FR); Emilie FARGIER, Compiègne (FR); Marie-Emmanuelle LE GUERN, Compiègne (FR); Marc VERLEYE, Remy (FR); Patrice TEWA, Noisly-le-Sec (FR); Xavier ZEISSER, Paris (FR)

(21) Appl. No.: 16/173,536

(22) PCT Filed: Apr. 27, 2017
(86) PCT No.: PCT/EP2017/060123

§ 371 Date: Oct. 29, 2018

(30) Foreign Application Priority Data
Apr. 27, 2016 (FR) 1670198

(51) Int. Cl. A61K 9/02; A61K 9/00; A61K 36/064; A61K 47/14; A61P 31/10; A61P 15/02

(52) U.S. Cl. CPC A61K 9/02; A61K 9/0034; A61P 15/02; A61K 47/14; A61P 31/10; A61K 36/064

(57) ABSTRACT

The present invention relates to a composition comprising a mixture of Saccharomyces boulardii cells and of a wax.

### FIELD OF THE INVENTION

The present invention relates to a composition that is useful for the preparation of a vaginal suppository and also to a medical device, to a medicament, or to a cosmetic product comprising said composition.

### TECHNICAL BACKGROUND

Vulvovaginal infections are low genital tract infections which affect the vulva and/or the vagina. They manifest themselves through an irritation, itching, pain and abnormal vaginal losses. These infections are very common. They are in fact the most common cause of medical consultation among women.

Vulvovaginal infections are mainly of bacterial or mycosal origin, parasitic infections, mainly with trichomonas, representing only approximately 10% of these infections.

Bacterial vulvovaginal infections represent approximately 50 to 60% of all infections of the vulva and vagina. The most common microorganisms encountered in these infections are Gardnerella vaginalis, Gardnerella mobiluncus, Chlamydia trachomatis, Mycoplasma hominis, Peptostreptococcus species and Ureaplasma urealyticum.

Approximately 70% of women experience a mycosal vulvovaginal infection during their lifetime. Moreover, mycosal infection is found in 40% of women carrying a bacterial vulvovaginal infection. Mycosal vulvovaginal infections are essentially candidiasis infections.

Bacterial and parasitic infections are mainly treated using antibiotics/antiparasitics of the nitroimidazole family, such as metronidazole, while mycosal infections are usually treated using antifungals of the family of imidazole-comprising agents, such as econazole, miconazole or fluconazole.

However, despite therapeutic treatment, approximately 5% of women experience a recurrent vulvovaginal infection, defined by more than four infections per year. In addition, half of the women treated for a bacterial vulvovaginal infection experience a recurrence within 12 months.

The treatments for vulvovaginal infections that are proposed are not therefore entirely satisfactory and it remains necessary to find alternative treatments, which make it possible in particular to limit recurrences.

### SUMMARY OF THE INVENTION

The present invention ensues from the unexpected demonstration, by the inventors, that it was possible to formulate vagial suppositories using a mixture of freeze-dried Saccharomyces boulardii yeast cells and of wax and that these vagial suppositories were capable of releasing live S. boulardii yeast cells at the level of the vaginal mucosa, which makes it possible to combat vulvovaginal infections, in particular by secretion of capric acid. Furthermore, antibiotic treatments for vulvovaginal infections generally lead to a depletion of the protective vaginal flora, which promotes recurrences and reinfections. The use of vagial suppositories according to the invention in the context of an antibiotic therapy therefore makes it possible to limit the risks of recurrence or of reinfection that would be associated with an imbalance of the saprophytic flora.

Thus, the present invention relates to a composition comprising, or consisting of, a mixture of Saccharomyces boulardii yeast cells and of a wax.

The present invention also relates to a medical device, to a medicament or to a cosmetic product comprising, or consisting of, a composition as defined above.

In one particular embodiment, the invention relates to the medical device or to the medicament as defined above, for use thereof as a vaginal suppository, for use thereof in the prevention or treatment of vulvovaginal infections or for use thereof in the re-balancing of the vaginal flora, in particular after treatment with a local or systemic anti-infective and/or for contributing to consolidating the recovery after having taken a local vaginal anti-infective treatment, and/or for preventing recurrences of vaginal infections.

The present invention also relates to the use of a composition as defined above, for the preparation of a vaginal suppository, of a medicament or of a medical device intended for the prevention or treatment of vulvovaginal infections, or intended for the re-balancing of the vaginal flora, in particular after treatment with a local or systemic anti-infective, and/or for contributing to consolidating the recovery after having taken a local vaginal anti-infective treatment, and/or for preventing recurrences of vaginal infections.

The present invention also relates to the cosmetic use of a composition as defined above or of a cosmetic product as defined above, for the intimate hygiene or personal hygiene of an individual.

The present invention also relates to a method for preventing or treating a vulvovaginal infection in an individual, and also to a method for re-balancing the vaginal flora, in particular after treatment with a local or systemic anti-infective, and/or for contributing to consolidating the recovery after having taken a local vaginal anti-infective treatment and/or for preventing recurrences of vaginal infections in an individual, in which method a composition as defined above is administered to the individual, especially in a prophylactically or therapeutically effective amount.

The present invention also relates to a method for the intimate hygiene or personal hygiene of an individual, in which method a composition as defined above or a cosmetic product as defined above is administered to the individual, especially in a cosmetically effective amount.

The present invention also relates to a method for obtaining a composition as defined above, comprising the following steps:
if necessary, the wax is heated until it is made liquid;
the wax is kept in the liquid state at a temperature below 42° C. and Saccharomyces boulardii yeast cells are added with stirring;
the mixture is left to cool with stirring while keeping it liquid up to a temperature below 36° C.;
the mixture is poured into a mould;
the mixture is removed from the mould after solidification.

### DETAILED DESCRIPTION OF THE INVENTION

As a preliminary, it will be recalled that the term “comprising” means “including”, “containing” or “encompassing”, that is to say that, when a subject “comprises” an element or several elements, elements other than those mentioned can also be included in the subject. Conversely, the expression “consisting of” means “constituted of”, that is to say that, when a subject “consists of” an element or several elements, the subject cannot comprise elements other than those mentioned.

### Saccharomyces Boulardii

Saccharomyces boulardii, abbreviated to S. boulardii, is a yeast well known to those skilled in the art. It is in particular described in Hennequin et al. (2001) J. Clin. Microbiol. 39:551-559. The nomenclatures “Saccharomyces boulardii” and “Saccharomyces cerevisiae var. boulardii” (abbreviated to S. cerevisiae var. boulardii) are intended herein to be considered equivalent.

Preferably, the Saccharomyces boulardii yeast cells according to the invention are obtained from the pharmaceutical products of the Bioflor®, Bioflora®, Codex®, Econorm®, Enflor®, Enterol®, Florastor®, Floratil®, Florestor®, Inteflora®, Perenterol®, Perenteryl®, Precosa®, Reflor®, Ultra-Levura® or Ultra-Levure® brand name. The Saccharomyces boulardii yeast cells according to the invention can also be identical to the deposit made with the American Type Culture Collection (ATCC, USA) under the reference 74012, with the Collection Nationale de Culture et de Microorganismes [French National Collection of Microorganisms and Culture] (CNCM, Institut Pasteur, France) under the reference I-745 or with the Centraalbureau voor Schimmelcultures [Dutch Fungal Biodiversity Centre] (CBS, The Netherlands) under the reference Hansen CBS 5926.

Preferably, the Saccharomyces boulardii yeast cells according to the invention are mixed with the wax in the form of a powder, which is in particular freeze-dried.

Advantageously, the viability or vitality of Saccharomyces boulardii yeast cells derived from freeze-dried material is greater than that which can be obtained with other methods of yeast cell preservation, such as drying for example.

The term “freeze-drying” is intended to mean herein a method of preservation in which live Saccharomyces boulardii yeast cells are frozen and are then subjected to sublimation of the frozen water that they contain, so as to give a freeze-dried material in the form of a dry yeast powder preferably containing less than 2% of water and more preferably less than 1% of water. Preferably, the freeze-dried Saccharomyces boulardii yeast cells are obtained from concentrated cells. Many freeze-drying methods well known to those skilled in the art can be used for producing freeze-dried Saccharomyces boulardii yeast cells according to the invention. However, it is preferred for the Saccharomyces boulardii yeast cells according to the invention to be freeze dried by means of the following freeze-drying process:
the Saccharomyces boulardii yeast cells are cultured in a liquid culture medium until the cells reach a stationary phase;
the Saccharomyces boulardii yeast cells are concentrated and the concentrate is frozen, often in the presence of a cryoprotective agent, such as lactose;
the concentrate is freeze-dried.

The freeze-dried Saccharomyces boulardii yeast cells are in the form of a powder.

Preferably, Saccharomyces boulardii is the only probiotic included in the mixture, the composition, the medicament, the medical device or the cosmetic product according to the invention. In other words, no other probiotic, such as bacteria of the Lactobacillus genus, in particular L. rhamnosus, L. fermentum, L. crispatus, L. jensinii or L. gasseri, or of the Enterococcus genus, in particular Enterococcus faecium, or yeasts of the Saccharomyces genus, in particular S. cerevisiae, is present in the mixture, the composition, the medicament, the medical device or the cosmetic product. As defined by the Food and Agriculture Organization (FAO) of the United Nations, a “probiotic” denotes a live (or viable) microorganism, such as a bacterium or a yeast, which, when it is administered in adequate amounts, confers a health benefit on the individual to whom it is administered.

Preferably, the mixture according to the invention comprises from 1.5×10⁹ to 2.5×10¹⁰, in particular from 2×10⁹ to 2×10¹⁰, colony forming units (CFUs) of Saccharomyces boulardii yeast cells.

Preferably, the mixture according to the invention comprises, or is constituted of, 15 to 35% (w/w) of Saccharomyces boulardii yeast cells in the form of a powder, which is in particular freeze-dried.

### Wax

The term “wax”, as intended herein, denotes a composition based on one or more esters of an alcohol, in particular glycerol, and of fatty acis, which is solid at ambient temperature (18-23° C.)

Preferably, the wax according to the invention is based on “hard fat”, within the meaning of the European Pharmacopoeia (Ph. Eur.), namely a mixture of triglycerides, of diglycerides and of monoglycerides, which can in particular be obtained either by esterification of glycerol with fatty acids of natural origin, or by transesterification of natural fats.

The fatty acids according to the invention are saturated or unsaturated, preferably saturated. Moreover, the fatty acids according to the invention preferably comprise at least 8 carbon atoms (C8) or at least 12 carbon atoms (C12). Likewise preferably, the fatty acids according to the invention comprise at most 18 carbon atoms (C18).

Preferably, the wax according to the invention comprises, or is based on, triglycerides, in particular saturated triglycerides. In particular, the invention comprises, or is based on, saturated C₈-C₁₈ or C₁₂-C₁₈ triglycerides.

Moreover, the wax according to the invention can comprise additives, such as at least one lubricant or at least one emulsifier, in particular selected from the group constituted of glyceryl monoricinoleate, of a polyethylene glycol monocetyl ether, in particular Ceteth-20, of a polyethylene glycol monostearyl ether, in particular Steareth-20, and of lecithin.

Preferably, the wax according to the invention has a melting point above 32° C. Likewise preferably, the wax according to the invention has a melting point below 37° C. More preferably, the wax according to the invention has a melting point above 32° C. and below 37° C.

As will clearly become apparent to those skilled in the art, the melting point is preferably measured under a normal pressure. As intended herein, a normal pressure is a pressure of 1 bar.

Preferably, the wax according to the invention is selected from the group constituted of suppocire® NA Pastilles (Gattefossé), Ovucire® 3460 Pastilles (Gattefossé) and Suppocire® AML Pastilles (Gattefossé).

Preferably, the wax according to the invention has a relative moisture content of 20% to 35% before mixing.

### Mixture

Preferably, the mixture according to the invention has a melting point above 32° C. Likewise preferably, the mixture according to the invention has a melting point below 37° C. More preferably, the mixture according to the invention has a melting point above 32° C. and below 37° C.

As will become clearly apparent to those skilled in the art, the melting point is preferably measured under a normal pressure. As intended herein, a normal pressure is a pressure of 1 bar.

Preferably, the mixture according to the invention has a liquefaction time of less than 10 min. in water at 37° C.

Preferably, the mixture according to the invention has a breaking strength greater than 5.4 kg at a temperature of 23° C.

Preferably, the mixture according to the invention comprises, or is constituted of, 15 to 35% (w/w) of Saccharomyces boulardii yeast cells in the form of a powder, which is in particular freeze-dried, and from 65 to 85% (w/w) of wax according to the invention. More preferably, the mixture according to the invention comprises, or is constituted of, 30% (w/w) of Saccharomyces boulardii yeast cells in the form of a powder, which is in particular freeze-dried, and 70% (w/w) of wax according to the invention.

Preferably, the composition, in particular pharmaceutical composition, according to the invention, the medical device according to the invention, the medicament according to the invention or the cosmetic product according to the invention comprises, or consists of, the mixture of a unit weight of 1 g to 5 g, in particular of approximately or exactly 2 g.

Likewise preferably, the composition, in particular pharmaceutical composition, according to the invention, the medical device according to the invention, the medicament according to the invention or the cosmetic product according to the invention comprises a unit weight of 0.15 g to 1.75 g, in particular of 0.3 to 0.7 g, of Saccharomyces boulardii yeast cells in the form of a powder, which is in particular freeze-dried. More preferably, the composition, in particular pharmaceutical composition, according to the invention, the medical device according to the invention, the medicament according to the invention or the cosmetic product according to the invention comprises a unit weight of 0.6 g of Saccharomyces boulardii yeast cells in the form of a powder, which is in particular freeze-dried.

### Applications

The composition according to the invention can be a pharmaceutical composition. In this case, the pharmaceutical composition can comprise one or more pharmaceutically acceptable excipients or carriers in addition to the mixture.

The vulvovaginal infections according to the invention are infections of the vulva and/or of the vagina. The vulvovaginal infections according to the invention may be:
bacterial infections, in particular with Gardnerella vaginalis, Gardnerella mobiluncus, Chlamydia trachomatis, Mycoplasma hominis, Peptostreptococcus species and Ureaplasma urealyticum,
mycosal infections, in particular with Candida, or parasitic infections, in particular with Trichomonas.

The anti-infectives according to the invention can in particular be:
antiseptics;
antibiotics, in particular of the nitroimidazole family, such metrodinasole;
antifungal agents, in particular of the family of imidazole-comprising agents, such as econazole, miconazole or fluconazole; or
antiparasitics, in particular of the nitromidazole family, such as metrodinazole.

The “re-balancing of the vaginal flora” as intended herein denotes the reestablishment of a normal or healthy vaginal flora, in particular in terms of diversity and of amount of vaginal microorganisms.

The composition according to the invention can be a cosmetic composition. In this case, the cosmetic composition can comprise one or more cosmetically acceptable excipients or carriers in addition to the mixture. Likewise, the cosmetic product according to the invention can comprise one or more cosmetically acceptable excipients or carriers in addition to the composition according to the invention.

The intimate hygiene or personal hygiene, as intended herein, denotes the hygiene of the vulva and/or of the vagina.

Preferably, the composition, in particular pharmaceutical composition, according to the invention, the medical device according to the invention, the medicament according to the invention or the cosmetic product according to the invention is administered vaginally, in particular in the form of a vaginal suppository.

The term “vaginal suppository”, as intended herein, is considered to be equivalent to “gynaecological suppository” or “suppository” or “ovule”.

Preferably, the individual according to the invention is a mammal, more particularly a human being, in particular of the female sex.

### EXAMPLE

Materials and Methods

1.1 Waxes

The various waxes used for the formulation of the vaginal suppositories incorporating Saccharomyces boulardii are described in the table below:

| Trade Name | | | |
|---|---|---|---|
| (Gattefossé) | Definition | Description | Application |
| Suppocire ® NA | Hard fat | Saturated C₁₂-C₁₈ semi- | Vaginally: mass for |
| Pastilles | EP/NF/JPE. | synthetic glycerides | ovules/suppositories |
| | | (triglycerides) | with a medium |
| | | Melting point | hydroxyl number (43 |
| | | 34.5-36.5° C. | mg KOH/g). |
| Ovucire ® 3460 | Hard Fat | Saturated C₁₂-C₁₈ semi- | Vaginally: mass for |
| Pastilles | EP/NF/JPE (and) | synthetic glycerides | ovules/suppositories |
| | glyceryl | (triglycerides) | with a high hydroxyl |
| | ricinoleate | containing additives | number (65 mg KOH/g) |
| | (and) | Melting point | |
| | Ceteth- | 32.5-34.0° C. | |
| | 20/Steareth-20 | | |
| | EP/NF. | | |
| Suppocire ® | Hard fat NF/JPE | Saturated C₈-C₁₈ semi- | Rectally: mass for |
| AML Pastilles | | synthetic glycerides | ovules/suppositories |
| | | (triglycerides) | with a low hydroxyl |
| | | containing a | number (6 mg KOH/g) |
| | | phospholipid | |
| | | (lecithin) | |
| | | Melting point | |
| | | 35.0-36.5° C. | |

1.2 Yeast

The yeast used is Saccharomyces boulardii in freeze-dried powder form (Ultra-Levure®, Biocodex, batch 7316).

1.3 Vaginal Suppository Production
1. The suppository mass is heated at 55° C. until it has completely melted.
2. The mixture is cooled to 40-41° C., with magnetic stirring, then the yeasts are added.
3. High-speed homogenization (9000 rpm) is carried out for one minute in order to ensure good dispersion of the yeasts in the suppository mass.
4. The stirring is then decreased and maintained at 500 rpm for the remainder of the cooling.
5. At 30-36° C., the mixture is poured into a 2 g mould with a ruler (the ideal temperature for pouring the vaginal suppositories was determined from a thermo-rheogram of the mixture carried out using a Rheomat 115 apparatus (LabMakelaar Benelux BV)).
6. The vaginal suppositories are cooled to ambient temperature for 4 minutes.
7. the vaginal suppositories are removed from the mould after 6 minutes at ambient temperature.

1.4 Physical Vaginal Suppository Characterization Test

Melting Point:

This method determines the temperature at which the vaginal suppository becomes sufficiently liquid to slide into a U-shaped capillary tube (1.5 mm internal diameter). This method is a simple and reliable means for comparing the melting points of the vaginal suppositories. This melting point must be below 37° C. Three vaginal suppositories are tested per batch.

Mechanical Strength:

This method determines the breaking strength of the vaginal suppositories. The value defined corresponds to the weight required to generate crushing thereof. The vaginal suppository is positioned on a jaw and connected to a rod to which weights are successively added. The force exerted on the vaginal suppository is increased until the vaginal suppository is crushed. The vaginal suppositories must be capable of withstanding a weight at least equal to 1800-2000 g. Five vaginal suppositories are tested per batch.

Liquefaction Time:

This method (reference Ph. Eur. 2.9.22 edition in force) determines the time required for vaginal suppository maintained in water at 37° C. to soften sufficiently for it to deform for a rigid rod to pass through it. This time must be less than 20 minutes. Three vaginal suppositories are tested per batch.

### Example 1

| | | |
|---|---|---|
| | | For 1 vaginal |
| | For 100 vaginal | suppository (unit |
| Formulation | suppositories | formulation) |

| | | | | |
|---|---|---|---|---|
| Yeast | 60 | g | 600 | mg |
| Suppocire ® | 140 | g | 1400 | mg |
| NA | | | | |
| Total | 200 | g | 2 000 | mg |

| Tests | Standards | Results |
|---|---|---|
| Mechanical | >1 kg 800-2 kg | >5.40 kg |
| strength | | |
| Liquefaction time | <20 minutes* | 14 min 16 s ± 100 s |
| Melting point | <37° C. | 35.4 ± 0.1° C. |
| CONCLUSION | | COMPLIES |

*Specifications of the European Pharmacopoeia, edition in force

### Example 2

| | | |
|---|---|---|
| | | For 1 vaginal |
| | For 100 vaginal | suppository (unit |
| Tests | suppositories | formulation) |

| | | | | |
|---|---|---|---|---|
| Yeast | 60 | g | 600 | mg |
| Ovucire ® | 140 | g | 1400 | mg |
| 3460 | | | | |
| Total | 200 | g | 2 000 | mg |

| Tests | Standards | Results |
|---|---|---|
| Mechanical strength | >1 kg 800-2 kg | >5.40 kg |
| Liquefaction time | <20 minutes* | 7 min 37 s ± 7 s |
| Melting point | <37° C. | 36.3 ± 0.1° C. |
| CONCLUSION | | COMPLIES |

*Specifications of the European Pharmacopoeia

### Example 3

| | | |
|---|---|---|
| | | For 1 vaginal |
| | For 100 vaginal | suppository (unit |
| Tests | suppositories | formulation) |

| | | | | |
|---|---|---|---|---|
| Yeast | 60 | g | 600 | mg |
| Suppocire ® | 140 | g | 1400 | mg |
| AML | | | | |
| Total | 200 | g | 2 000 | mg |

| Tests | Standards | Results |
|---|---|---|
| Mechanical strength | >1 kg 800-2 kg | >5.40 kg |
| Liquefaction time | <20 minutes* | 8 min 14 s ± 2 s |
| Melting point | <37° C. | 36.0 ± 0.1° C. |
| CONCLUSION | | COMPLIES |

*Specifications of the European Pharmacopoeia

1. A vaginal suppository comprising a composition comprising a mixture of Saccharomyces boulardii yeast cells and of a wax.

2. The vaginal suppository according to claim 1, in which the mixture has a melting point above 32° C. and below 37° C., under a normal pressure.

3. The vaginal suppository according to claim 1, in which the mixture has a liquefaction time of less than 10 min, in water at 37° C.

4. The vaginal suppository according to claimc 1, in which the mixture has a breaking strength of greater than 5.4 kg at a temperature of 23° C.

5. The vaginal suppository according to claim 1, in which the wax has a relative moisture content of 20% to 35% before mixing.

6. The vaginal suppository according to claim 1, in which the wax is based on saturated C₈-C₁₈ or C₁₂-C₁₈ triglycerides.

7. The vaginal suppository according to claim 1, in which the wax is selected from the group constituted of Suppocire® NA Pastilles (Gattefossé), Ovucire® 3460 Pastilles (Gattefossé) and Suppocire® AML Pastilles (Gattefossé).

8. The vaginal suppository according to claim 1, in which the Saccharomyces boulardii yeast cells are mixed in the form of a powder, which is in particular freeze-dried.

9. The vaginal suppository according to claim 1, in which the mixture is formed of 15 to 35% (w/w) of Saccharomyces boulardii yeast cells in powder form and of 65 to 85% (w/w) of wax.

10. The vaginal suppository according to claim 1, in which the mixture comprises from 1.5×10⁹ to 2.5×10¹⁰ colony forming units (CFUs) of Saccharomyces boulardii yeast cells.

11.-19. (canceled)

20. The vaginal suppository according to claim 1 comprising the mixture in a unit weight of 1 g to 5 g.

21. A method for preventing or treating vulvovaginal infections in an individual, comprising administering to the individual at least one vaginal suppository comprising a composition comprising a mixture of Saccharomyces boulardii yeast cells and of a wax.

22. A method for re-balancing of the vaginal flora, in particular after treatment with a local or systemic anti-infective, and/or for contributing to consolidating the recovery after having taken a local vaginal anti-infective treatment, and/or for preventing recurrences of vaginal infections in an individual comprising administering to the individual at least one vaginal suppository comprising a composition comprising a mixture of Saccharomyces boulardii yeast cells and of a wax.

23. A method for obtaining a vaginal suppository comprising a composition comprising a mixture of Saccharomyces boulardii yeast cells and of a wax, comprising the following steps:
if necessary, the wax is heated until it is made liquid;
the wax is kept in the liquid state at a temperature below 42° C. and Saccharomyces boulardii yeast cells are added with stirring;
the mixture is left to cool, with stirring, while keeping it liquid, to a temperature below 36° C.;
the mixture is poured into a mould; and
the mixture is removed from the mould after solidification thereby yielding the vaginal suppository.
